(19) United States
(12) Patent Application Publication
(10) Pub. No.: US 2008/0161427 A1
(43) Pub. Date: Jul. 3, 2008

(54) TRANSPORTABLE GAS-TO-LIQUIDD PLANT

(75) Inventors: Kenneth Agee, Bixby, OK (US); Juan Inga, Sapulpa, OK (US); John Hutton, Camberly Surrey (GB); Ed Sheehan, Tulsa, OK (US); Ad van Loenhout, Leiden (NL); Linda Zeelenberg, Den Haag (NL); Arjan Gerritse, Rotterdam (NL); Peter van Sloten, Voorhout (NL)

Correspondence Address: BAKER & MCKENZIE LLP, Pennzoil Place, South Tower, 711 Louisiana, Suite 3400, HOUSTON, TX, 77002-2716, US

(73) Assignee: SYNTROLEUM CORPORATION, TULSA, OK (US)

(21) Appl. No.: 11/855,619

(22) Filed: Sep. 14, 2007

Related U.S. Application Data
(62) Division of application No. 11/353,916, filed on Feb. 14, 2006, now abandoned.
Application No. 11/353,916 is a continuation-in-part of application No. 11/088,287, filed on Mar. 24, 2005, now abandoned.
(60) Provisional application No. 60/557,638, filed on Mar. 30, 2004.

(51) Int. Cl. C07C 2/00

(52) U.S. Cl. 518/700

(57) ABSTRACT

A transportable GTL processing facility constructed on an inland barge is provided. Also provided is a process for producing liquid hydrocarbons from natural gas utilizing a transportable GTL processing facility. The facility and process may be used to access and convert stranded natural gas in an economical fashion into liquid hydrocarbons. Further provided is a transportable GTL processing facility and process for producing liquid hydrocarbons wherein the liquid hydrocarbons are upgraded into transportation fuels and other locally usable materials. Water facilities of the transportable GTL processing facility are supplied from the sea near the barge.

### CROSS REFERENCE TO RELATED APPLICATIONS

This application is a continuation-in-part application of U.S. Ser. No. 11/088,287 filed on Mar. 24, 2005 which claimed priority to Provisional Application Ser. No. 60/557,638, filed on Mar. 30, 2004, the disclosures of which are incorporated herein by reference.

### FEDERALLY SPONSORED RESEARCH

The present invention was developed with funds from the Department of Defense. Therefore, the United States Government may have certain rights in the invention.

### REFERENCE TO MICROFICHE APPENDIX

Not applicable.

### FIELD OF THE INVENTION

This invention relates to gas-to-liquid (GTL) technology, and more particularly to GTL processes practiced on a mobile or transportable platform. The invention further relates to a transportable GTL facility which is capable of accessing stranded gas reserves.

### BACKGROUND

Of the estimated 5,500 TCF of natural gas reserves worldwide, nearly one-half is stranded, with over 50% of those reserves being offshore. For our purpose, the term “stranded gas” means natural gas that cannot be economically delivered to market using current gas transportation methods or current commercial GTL processes. Stranded gas includes associated and flared/vented gas, and gas that is re-injected purely for regulatory compliance rather than for reservoir-pressure maintenance. Some of the factors that determine when a pipeline is profitable include resource volume, transport route, pipeline distance, regulatory environment, market size and demand growth. Excess reserves may be considered stranded where a paltry delivery rate is required to avoid oversupply of local markets. Negative economics may also arise from technical complexity or expense associated with recovering and/or gathering the gas.

One method of producing stranded gas is to process it through a Fischer-Tropsch (FT) gas-to-liquid (GTL) system. GTL is an application of the basic Fischer-Tropsch (FT) process, wherein synthesis gas (or syngas, which is composed primarily of H₂ and CO) is reacted in the presence of a Fischer-Tropsch catalyst to produce heavier hydrocarbons. Possible Fischer-Tropsch end products include kerosene, naphtha, waxes, liquid paraffins and lubes, synthetic diesel, gasoline, and jet fuel. Stranded natural gas may be used as a raw feedstock for GTL operations, thereby monetizing otherwise worthless gas.

### SUMMARY OF THE INVENTION

The GTL barge provided by the invention is designed to develop natural gas assets in the 0.5-5.0 TCF range where there is currently no infrastructure to produce and transport the stranded reserves since the fields are not large enough to economically support an LNG facility. By employing the barge, the owner/operator of the field gets the added benefit of being able to book the reserves. The GTL barge includes a syngas generating section and a Fischer-Tropsch (FT) reaction section. The GTL barge is an inland barge and, therefore, not ocean-going. The GTL barge is designed to be transportable to or near a gas formation by lift ship or other dry haul method. Product upgrading may also be included in the GTL barge, either integrated on the GTL inland barge or located near the GTL barge, such as on a separate barge or on-shore.

### DETAILED DESCRIPTION OF THE INVENTION

The transportable GTL inland barge enables an exploration and production company to produce and thus monetize stranded gas fields. The GTL barge focuses on gas reserves in or near shallow water or onshore gas reserves that are near the coastline or other navigable waterway.

The GTL barge is ideally suited to process associated rich gas that might otherwise be flared or re-injected. Estimated worldwide flared gas is about 10 billion ft³ per day. A single GTL barge, for example, may be designed to produce approximately 20,000 barrels per day (bpd) of total liquid products, including approximately 12,000 bpd of GTL products. Assuming the natural gas has about 2 gpm propane and higher carbon number natural gas liquid (“NGL”); the combined NGL and GTL products are about 8700 bpd of clean diesel fuel, 7300 bpd of naphtha, and 4400 bpd of LPG. The mobility and/or transportability of a GTL plant enables the operator to mitigate long-term project and financial risk by having the ability to relocate the barge. The GTL barges may be constructed in shipyards.

The GTL barge is used where it is within a distance from a gas reserve to-which it would be economically feasible to build a pipeline to transport natural gas feed to the barge. The products from the barge may be either synthetic crude or upgraded products, including for example, transportation fuels.

Where there are two or more reserves located fairly close to each other, the reserves may be accumulated by pipeline or by compressed natural gas (“CNG”) to supply feedstock to a single GTL barge. Where two or more barges are located in a region, a single syncrude upgrading section may serve such barges and the upgrading section may be located on one of the barges, a separate barge or a separate location. A shuttle barge may be used to carry syncrude to the product upgrading unit.

In one embodiment of the invention, the GTL barge is constructed on an inland barge. As defined herein, the term “inland barge” means a barge which is transportable by lift ship or other form of barge dry haul and which is not suitable for towing or operation at sea or in any waters having wave action greater than that of Sea State 0 (as defined by Pierson-Moskowitz Sea Spectrum). It should be noted that the Sea State 0 is based upon wind speeds of around three (3) knots. However, as used herein, the term inland barge will include designs which may withstand wind loads of about 120 kilometers per hour or greater. The inland barge, however, may be towed within inland waters, such as rivers, lakes and intercoastal waterways. The inland barge is installed and then operated only in calm water. “Installed” is defined as either freely floating in confining moorings or fixed in a non-floating arrangement. Confining moorings will allow the barge to float on a water body allowing only uniform vertical motion with essentially no lateral or twisting motion. In some embodiments, a barge having jacking legs may be used and installation of the jacking legs installs the barge. As used herein, the term “calm water” means near shore, such as on pylons, beached, or in a natural or man-made inlet which may or may not be dammed and/or drained, or on a fixed platform if off-shore. The term “calm water” may also include inland waterways, such as rivers, lakes, ship channels, bayous, and intercoastal waterways which are protected from substantial natural wave action. Other methods of securing the barge in calm water include use of a flotation jacket surrounding the outer perimeter of the barge, anchoring, or installation of legs under the barge. As used herein, the terms “GTL barge” and “GTL inland barge” are synonymous.

As the term is defined herein, inland barges are not intended for offshore use unless installed on a fixed platform. Similarly, the term “inland barge” does not include ocean-going vessels which are mobile under their own power. Rather, the inland barge is transported via dry haul lift ship to a location within a commercially practical distance from an appropriate natural gas reserve. Commercially practical distances are those in which a pipeline from the reserve to the barge may be constructed while maintaining the total cost of synthetic crude or hydrocarbon product production within competitive market limits. Such distances vary according to the structure of the intervening terrain as well as other production and market factors, such as then current market prices for the hydrocarbon products to be produced and local labor costs.

The GTL barge may be split into numerous sections, for example, a natural gas purification section, a natural gas liquid recovery section; a syngas production section; a Fischer-Tropsch Reaction (“FTR”) section; and a product separation/upgrading section. These sections may or may not be modules as equipment from one section may be intermingled with equipment from another section. Alternatively, each section may be substantially self-contained and located substantially separately from the other sections

For the production of syngas, two basic methods have been employed. The two methods are steam reforming, wherein one or more light hydrocarbons such as methane are reacted with steam over a catalyst to form carbon monoxide and hydrogen, and partial oxidation, wherein one or more light hydrocarbons are combusted or reacted sub-stoichiometrically to produce synthesis gas.

The basic steam reforming reaction of methane is represented by the following formula:

CH₄+H₂O+Catalyst→CO+3H₂

The steam reforming reaction is endothermic and a catalyst containing nickel is often utilized. The hydrogen to carbon monoxide ratio of the synthesis gas produced by steam reforming of methane is approximately 3:1.

Partial oxidation is the non-catalytic, sub-stoichiometric combustion of light hydrocarbons such as methane to produce the synthesis gas. The basic reaction is represented as follows:

CH₄+½O₂→CO+2H₂

The partial oxidation reaction is typically carried out using high purity oxygen. High purity oxygen can be quite expensive. The hydrogen to carbon monoxide ratio of synthesis gas produced by the partial oxidation of methane is approximately 2:1.

In some situations these approaches may be combined. A combination of partial oxidation and steam reforming, known as autothermal reforming, wherein air is used as a source of oxygen for the partial oxidation reaction has also been used for producing synthesis gas heretofore. Autothermal reforming is a combination of partial oxidation and steam reforming where the exothermic heat of the partial oxidation supplies the necessary heat for the endothermic steam reforming reaction. The autothermal reforming process can be carried out in a relatively inexpensive refractory lined carbon steel vessel whereby low cost is typically involved.

The autothermal process generally results in a lower hydrogen to carbon monoxide ratio in the synthesis gas than does steam reforming alone. That is, as stated above, the steam reforming reaction with methane results in a ratio of about 3:1 while the partial oxidation of methane results in a ratio of about 2:1. The optimum, ratio for the hydrocarbon synthesis reaction carried out at low or medium pressure over a cobalt catalyst is 2:1. When the feed to the autothermal reforming process is a mixture of light hydrocarbons such as a natural gas stream, some form of additional control is desired to maintain the ratio of hydrogen to carbon monoxide in the synthesis gas at the optimum ratio of about 2:1.

In some embodiments the syngas production section of the GTL barge is an Autothermal Reforming unit (“ATR”). The ATR section is any capable of producing a synthesis gas to be utilized in the associated Fischer-Tropsch reaction section. As will be understood in the art, ATR may take different forms but generally is comprised of a vessel having a reforming catalyst (e.g. nickel-containing catalyst) therein which converts the air/steam/natural gas to a synthesis gas. Syngas useful in producing a Fischer-Tropsch product may contain hydrogen, carbon monoxide and nitrogen with H₂:CO ratios from about 0.8:1 to about 3.0:1. Operating conditions and parameters of an autothermal reactor for producing a syngas useful in the process of the invention are well known to those skilled in the art. Such operating conditions and parameters include but are not limited to those disclosed in U.S. Pat. No. 6,155,039, and U.S. Provisional Patent Application Ser. No. 60/497,177.

In some embodiments of the invention, an autothermal reforming process is utilized wherein the ATR is fed natural gas and air-derived oxygen. The term “air-derived oxygen” as used herein refers to oxygen obtained from air by means other than a cryogenic air separation plant. For example, air may be passed through a selective membrane through which oxygen is selectively absorbed and/or passed. Such membranes are known in the art, for example, in U.S. Pat. No. 6,406,518. Included in such membranes are those commonly referred to as mixed conductor ceramic membranes, oxygen ion transport membranes, and ionic/mixed conductor membranes.

The syngas may be optionally preheated before it is delivered to the Fischer-Tropsch reactor. As will be understood in the art, Fischer Tropsch reactors are well known in the art and basically are comprised of a vessel containing an appropriate catalyst (e.g. cobalt-containing catalyst) therein. Fischer-Tropsch catalysts include, for example, cobalt, iron, ruthenium as well as other Group IVA, Group VIII and Group VIIB transition metals or combinations of such metals, to prepare both saturated and unsaturated hydrocarbons. There are several known catalysts which are used in converting a synthesis gas depending on the product desired; e.g., see U.S. Pat. Nos. 6,169,120 and 6,239,184. The Fischer-Tropsch catalyst may include a support, such as a metal-oxide support, including for example, silica, alumina, silica-alumina or titanium oxides. For example, a cobalt (Co) catalyst on transition alumina may be used. The Co concentration on the support may be between about 5 wt % and about 40 wt %. Certain catalyst promoters and stabilizers, which are known in the art, may optionally be used. Stabilizers include Group IIA or Group IIIB metals, while the promoters may include elements from Group IVA, Group VIII or Group VIIB. The Fischer-Tropsch catalyst and reaction conditions may be selected to be optimal for desired reaction products, such as for hydrocarbons of certain chain lengths or number of carbon atoms. Any of the following reactor configurations may be employed for Fischer-Tropsch synthesis: fixed bed, slurry bed reactor, ebullating bed, fluidizing bed, or continuously stirred tank reactor (“CSTR”). The FTR may be operated at a pressure from about 100 psia to about 800 psia and a temperature from about 300° F. to about 600° F. The reactor gas hourly space velocity (“GHSV”) may be from about 1000 hr⁻¹ to about 15000 hr⁻¹. Operating conditions and parameters of the FTR useful in the process of the invention are well known to those skilled in the art. Such operating conditions and parameters include but are not limited to those disclosed in U.S. Pat. No. 6,172,124.

Given the safety issues of dealing with pure oxygen, air based systems have a significant advantage with a mobile or transportable system. The use of air instead of pure oxygen for generating synthesis gas in a mobile or transportable process wherein hydrocarbon processing equipment is necessarily located in relatively close proximity to any air or oxygen-handling equipment significantly raises safety, may be less capital-intensive and may reduce the size of the plant and facilities.

The product separation/upgrading section includes equipment for processing the syncrude products from the FT section to fuel-grade products, namely diesel and naphtha. The upgrading equipment may be installed on the GTL barge or may be located on an adjacent platform, barge or onshore facility. Preferably, products are not stored on the barge but rather transported to a separate location, such as a floating storage offloading unit (FSO) farther out from the shore to hold the product. The FSO may be a reconditioned single hull tanker. Product upgrading equipment may include distillation tower(s) as well as hydroprocessing and hydrocracking reactors.

The utilities section supplies the utilities for all the processes. An integrated utility facility will provide the most synergy of the various utilities needed for the processes. Some common utilities include, but are not limited to, air, nitrogen, electric power generation, fuel gas system, flare systems, drain systems, boiler feed water supply, steam generation and cooling water. Other utilities include, but are not limited to, hydrogen generation, propane refrigeration, catalyst handling and storage/offloading of multiple products, depending on the GTL barge configuration.

In a preferred embodiment, the flare is a ground flare and may be located on an auxiliary deck or separate from the GTL barge, such as on shore or on a separate barge or platform.

Cooling on the GTL barge may come from a combination of a closed-loop cooling water system, high pressure BFW heat recovery, air coolers and direct seawater cooling. Water for the utilities can come from a variety of sources, but most likely would be drawn from the sea near the barge. In a preferred embodiment, the sea water would be drawn from about 250 to about 1750 feet below the surface. Sea water may also be drawn from depths from about 50, 100 or 200 feet below the surface. At this depth, the water is typically about 35 to about 55° F. The sea water preferably has a temperature of ≦55° F., more preferably ≦50° F. In alternate embodiments, the sea water is in tropical climates and the temperature difference of the sea water and the surface of the sea may be as little as about 5.4° F. The amount of deep sea water is dependent upon the temperature of the source water, the discharge temperature, and the heat removal requirements of the GTL process equipment.

Typically, water used for cooling and discharged to the surface of the sea is limited to about 5.4° F. above the surface temperature, depending on local environmental regulation. For example, deep sea water that starts out at 40° F. can be heated to the local surface temperature plus a margin which is determined by the location. For 90° F. surface water, the discharge temperature could be 95.4° F., resulting in the deep sea water being heated 55.4° F. versus surface water that could only be heated 5.4° F. The increased heat capacity of the deep sea water reduces the amount of water by approximately 1025%. Deep sea water usage results in considerable savings in sea water pumps, sea water piping, heat exchanger surface area, and consumption of power.

Colder water may also improve contaminate removal from syngas, increase FT catalyst activity, reduced FT catalyst consumption, increase product recovery, reduce compression power requirements, reduce process piping sizes, and reduce FT reactor cooling coils.

Sea water intake lines would deliver the deep sea water to the barge via a moon pool located within the vessel hull. The moon pool would feed the sea water pumps circulating water to process and utility equipment. Water is forced into the moon pool by the hydraulic head exerted by the surrounding water. In alternate embodiments, any mechanism that is capable of supplying sea water to the cooling water system may be used.

A GTL plant typically produces and requires large quantities of high grade energy (High Pressure (HP) steam) and produces an excess of lower grade energy (Medium pressure (MP) steam and low-BTU tail gas). By balancing the output from the GTL section with the input needed to produce the syngas, HP steam generated by the GTL section is used as a feed stream to the reformer and can be used to drive compressors and to produce power. All the tail gas is used for process heating and additional HP steam generation. Some additional fuel gas is required for electrical power generation, low-BTU combustors and direct fired heaters.

A central power plant offers flexibility via possible load shedding of non-essential consumers, load sharing between spare generators and thus higher availability of the main processes. Combinations of steam turbines, gas turbines and generators may be used to provide power. In a preferred embodiment, a steam turbine along with a gas turbine and two diesel engine generators are used to provide power.

The GTL barge requires hydrogen, which is produced in the reformer of the GTL process. In a preferred embodiment, a separate steam methane reformer (SMR) is proposed to supply hydrogen during startup. In alternate embodiments, a pressure swing absorber (PSA) may also be used alone or in tandem with the SMR.

While the invention has been described with respect to a limited number of embodiments, the specific features of one embodiment should not be attributed to other embodiments of the invention. No single embodiment is representative of all aspects of the inventions. Moreover, variations and modifications therefrom exist. For example, the GTL barge described herein may comprise other components. The appended claims intend to cover all such variations and modifications as falling within the scope of the invention.

We claim:

1-9. (canceled)

10. A process for producing synthetic liquid hydrocarbons from natural gas at or near calm water comprising the steps of:
dry hauling a mobile synthetic liquid hydrocarbon production facility to or near the calm water location;
installing the mobile hydrocarbon production facility so that it is not freely floating;
connecting the mobile hydrocarbon production facility to a source of natural gas; and
synthesizing heavier hydrocarbons from the natural gas.

11. The process of claim 6 further wherein the transportable liquid hydrocarbon facility further comprises a utilities unit, wherein the utilities system comprises a cooling water system that is supplied with deep sea water.

12. The process of claim 11 wherein the sea water is <55° F.

13. The process of claim 11 wherein the sea water is <50° F.

14. The process of claim 11, wherein the temperature difference of the sea water and the surface of the sea is about 5.4° F.

15. The process of claim 11 wherein the sea water is taken from a depth of >250 feet.

16. The process of claim 11 wherein the sea water is taken from a depth of >200 feet.

17. The process of claim 11 wherein the sea water is taken from a depth of >100 feet.

18. The process of claim 11 wherein the sea water is taken from a depth of >50 feet.
